(19) United States
(12) Patent Application Publication
(10) Pub. No.: US 2026/0013743 A1
(43) Pub. Date: Jan. 15, 2026

(54) SETPOINT ADJUSTMENT FOR ARTERIAL PRESSURE MEASUREMENTS

(71) Applicant: Becton, Dickinson and Company, Franklin Lakes, NJ (US)

(72) Inventors: Boris Reuderink, Amersfoort (NL); Jeroen Van Goudoever, Amstelveen (NL); Hans Jean Paul Kuijkens, Amsterdam (NL)

(21) Appl. No.: 19/333,955

(22) Filed: Sep. 19, 2025

Related U.S. Application Data
(63) Continuation of application No. PCT/US2024/020723, filed on Mar. 20, 2024.
(60) Provisional application No. 63/492,182, filed on Mar. 24, 2023.

(51) Int. Cl. A61B 5/0225; A61B 5/00; A61B 5/021; A61B 5/022

(52) U.S. Cl. CPC A61B 5/02255; A61B 5/02116; A61B 5/7267; A61B 5/7271; A61B 5/02241

(57) ABSTRACT

A method of measuring arterial pressure includes receiving a first arterial volume setpoint, adjusting an air pressure of an air bladder to a first air pressure, receiving a first plethysmographic signal from a plethysmographic sensor, analyzing the first plethysmographic signal to determine a first waveform feature, generating a setpoint adjustment value based on the first waveform feature, and generating a second arterial volume setpoint. The first arterial volume setpoint is received by a processor and an air pressure of the air bladder is adjusted by a pressure controller operatively connected to the processor. The first plethysmographic signal is representative of a first arterial volume waveform and is received by the processor while the air pressure of the air bladder is at the first air pressure. The second arterial volume setpoint is generated by adjusting the first arterial volume setpoint based on the setpoint adjustment value.

### CROSS-REFERENCE TO RELATED APPLICATION(S)

This application is a continuation of International Application No. PCT/US2024/020723, filed Mar. 20, 2024, and entitled “SETPOINT ADJUSTMENT FOR ARTERIAL PRESSURE MEASUREMENTS,” which claims the benefit of U.S. Provisional Application No. 63/492,182, filed Mar. 24, 2023, and entitled “SETPOINT ADJUSTMENT FOR ARTERIAL PRESSURE MEASUREMENTS,” the disclosures of which are hereby incorporated by reference in their entirety.

### BACKGROUND

The present disclosure relates to arterial pressure sensing, and more particularly to setpoint adjustment for arterial pressure measurements using a volume clamping technique.

Some non-invasive arterial pressure sensors generate a pressure reading by clamping (i.e., holding constant) arterial volume using plethysmographic readings. A volume setpoint and a fast servo system are used to make arterial pressure waveform measurements using a volume clamp technique. However, it can be difficult to acquire the proper setpoint, which can lead to inaccurate arterial pressure waveform data. One approach to identifying the correct setpoint is known as physical calibration (PHYSIOCAL), as described in U.S. Pat. No. 4,510,940.

### SUMMARY

An example of a method of measuring arterial pressure includes receiving a first arterial volume setpoint, adjusting an air pressure of an air bladder to a first air pressure, receiving a first plethysmographic signal from a plethysmographic sensor, analyzing the first plethysmographic signal to determine a first waveform feature, generating a setpoint adjustment value based on the first waveform feature, and generating a second arterial volume setpoint. The first arterial volume setpoint is received by a processor and an air pressure of the air bladder is adjusted by a pressure controller operatively connected to the processor. The first plethysmographic signal is representative of a first arterial volume waveform and is received by the processor while the air pressure of the air bladder is at the first air pressure. The second arterial volume setpoint is generated by adjusting the first arterial volume setpoint based on the setpoint adjustment value.

An example of a system for measuring arterial pressure includes a plethysmographic sensor configured to sense arterial volume, a pressure controller pneumatically connected to an air bladder and configured to adjust an air pressure of the airbladder, a processor in operable communication with the pressure controller and the plethysmographic sensor, and a memory encoding instructions. The instructions, when executed, cause the processor to receive a first arterial volume setpoint, cause the pressure controller to adjust an air pressure of the air bladder to a first air pressure, receive a first plethysmographic signal from the plethysmographic sensor while the air bladder is at the first air pressure analyze the first plethysmographic signal to determine a first waveform feature, generate a setpoint adjustment value based on the first waveform feature, and generate a second arterial volume setpoint by adjusting the first arterial volume setpoint by the setpoint adjustment value. The first plethysmographic signal representative of a first arterial volume waveform while the air pressure of the air bladder is at the first air pressure.

The present summary is provided only by way of example, and not limitation. Other aspects of the present disclosure will be appreciated in view of the entirety of the present disclosure, including the entire text, claims, and accompanying figures.

### BRIEF DESCRIPTION OF THE DRAWINGS

FIG. 1 is a schematic diagram of an example of a system for sensing arterial pressure.

FIG. 2 is an isometric view of an example of a non-invasive sensor suitable for use with the system of FIG. 1.

FIG. 3 is a flow diagram of an example of a method of performing a volume clamp measurement.

FIG. 4 is a parallel graph of examples of air pressure signals and plethysmographic signals.

FIG. 5 is a flow diagram of an example of a method of determining plethysmographic setpoint.

FIG. 6 is a graph illustrating a plethysmographic waveform.

While the above-identified figures set forth one or more examples of the present disclosure, other examples are also contemplated, as noted in the discussion. In all cases, this disclosure presents the invention by way of representation and not limitation. It should be understood that numerous other modifications and examples can be devised by those skilled in the art, which fall within the scope and spirit of the principles of the invention. The figures may not be drawn to scale, and applications and examples of the present invention may include features and components not specifically shown in the drawings.

### DETAILED DESCRIPTION

The present disclosure describes an approach for determining plethysmographic setpoints used for volume-clamped arterial pressure measurements. More specifically, the present disclosure describes systems and methods for creating and using setpoint adjustment values that can be used to adjust an existing or previously-used plethysmographic setpoint. Existing methods of setpoint determination ignore the value of the previous setpoint in determining a new setpoint, potentially leading to persistent or repeated errors in setpoint determination. The methods and systems described herein determine a setpoint based on a previously-used setpoint and a setpoint adjustment value. The setpoint adjustment values are based on waveform features of an arterial volume waveform measured at a constant applied pressure.

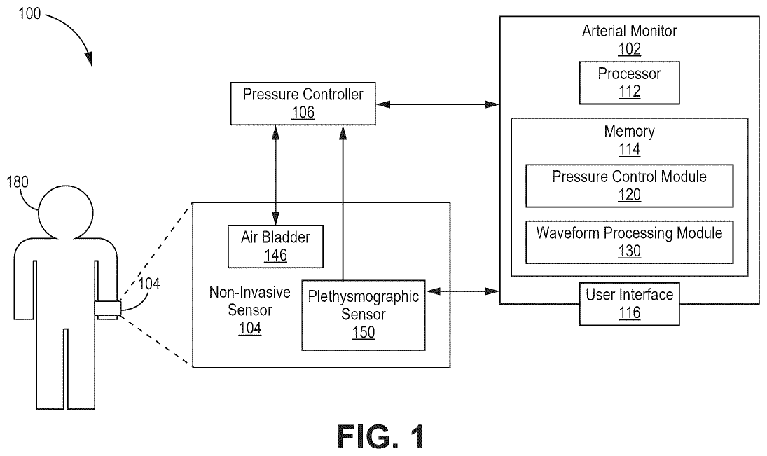

FIG. 1 is a schematic diagram of hemodynamic sensing system 100, which is an example of a system for sensing and using hemodynamic data. System 100 includes arterial monitor 102, non-invasive sensor 104, and air pressure controller 106. Arterial monitor 102 includes processor 112, memory 114, and user interface 116. Memory 114 stores pressure control module 120 and setpoint adjustment module 130. Non-invasive sensor 104 includes air bladder 146 and plethysmographic sensor 150. FIG. 1 also depicts patient 180, who is shown as wearing non-invasive sensor 104. Hemodynamic sensing system 100 is configured to perform one or more methods described herein. Hemodynamic sensing system 100 is configured to sense and use hemodynamic data, such as arterial volume data and/or arterial pressure data. More generally, hemodynamic sensing system 100 is configured to perform any of the functions attributed herein to a hemodynamic sensor or a hemodynamic sensing system, including receiving an output from any source referenced herein, detecting any condition or event referenced herein, and generating and providing data and information as referenced herein.

Processor 112 can execute software, applications, and/or programs stored on memory 114. Examples of processor 112 can include one or more of a processor, a microprocessor, a controller, a digital signal processor (DSP), an application specific integrated circuit (ASIC), a field-programmable gate array (FPGA), or other equivalent discrete or integrated logic circuitry. Processor 112 can be entirely or partially mounted on one or more circuit boards.

Memory 114 is configured to store information and, in some examples, can be described as a computer-readable storage medium. Memory 114, in some examples, is described as computer-readable storage media. In some examples, a computer-readable storage medium can include a non-transitory medium. The term “non-transitory” can indicate that the storage medium is not embodied in a carrier wave or a propagated signal. In certain examples, a non-transitory storage medium can store data that can, over time, change (e.g., in RAM or cache). In some examples, memory 114 is a temporary memory. As used herein, a temporary memory refers to a memory having a primary purpose that is not long-term storage. Memory 114, in some examples, is described as volatile memory. As used herein, a volatile memory refers to a memory that the memory does not maintain stored contents when power to the memory 114 is turned off. Examples of volatile memories can include random access memories (RAM), dynamic random access memories (DRAM), static random access memories (SRAM), and other forms of volatile memories. In some examples, the memory is used to store program instructions for execution by the processor. The memory, in one example, is used by software or applications running on hemodynamic sensing system 100 (e.g., by a computer-implemented machine learning model or a data processing module) to temporarily store information during program execution.

Memory 114, in some examples, also includes one or more computer-readable storage media. Memory 114 can be configured to store larger amounts of information than volatile memory. Memory 114 can further be configured for long-term storage of information. In some examples, memory 114 includes non-volatile storage elements. Examples of such non-volatile storage elements can include, for example, magnetic hard discs, optical discs, floppy discs, flash memories, or forms of electrically programmable memories (EPROM) or electrically erasable and programmable (EEPROM) memories.

User interface 116 is an input and/or output device and enables an operator to control operation of hemodynamic sensing system 100. For example, user interface 116 can be configured to receive inputs from an operator and/or provide outputs. User interface 116 can include one or more of a sound card, a video graphics card, a speaker, a display device (such as a liquid crystal display (LCD), a light emitting diode (LED) display, an organic light emitting diode (OLED) display, etc.), a touchscreen, a keyboard, a mouse, a joystick, or other type of device for facilitating input and/or output of information in a form understandable to users and/or machines.

Non-invasive sensor 104 is a wearable and non-invasive sensor for sensing hemodynamic parameters. More specifically, non-invasive sensor 104 is able to sense hemodynamic parameters using air bladder 146 and plethysmographic sensor 150, and does not require physically-invasive techniques for operation. Non-invasive sensor 104 is configured to sense both arterial pressure waveforms and arterial volume waveforms using a combination of air bladder 146 and plethysmographic sensor 150, as will be explained in more detail subsequently.

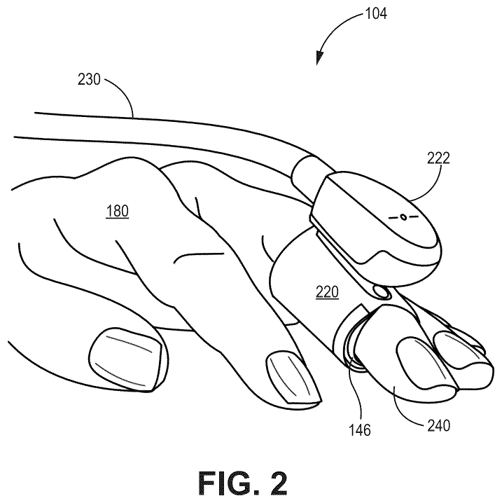

Air bladder 146 is an annular and pressurizable bladder capable of applying variable mechanical pressure to an appendage of patient 180. As will be explained in more detail subsequently, the pressure of air bladder 146 can be adjusted by air pressure controller 106. Air bladder 146 is deformable such that as the air pressure of air bladder 146 is adjusted, air bladder 146 expands and exerts force on the appendage of patient 180, constricting arterial volume in the appendage. The portion of the arterial volume of the appendage of patient 180 surrounded circumferentially by air bladder 146 defines a clamped volume when air bladder 146 applies or exerts a force on the appendage. Air bladder 146 is generally re-positionable on appendages of patient 180. The force exerted by air bladder 146 can be referred to as a “clamping force” in some examples. As will be described subsequently with respect to FIG. 2, air bladder 146 can be formed as an annular cuff that can be disposed circumferentially around a finger of patient 180, such that air bladder 146 circumferentially surrounds the finger of patient 180 when worn and can apply pressure to one or more arteries of the finger located circumferentially within the annular cuff. As air is flowed into the air bladder 146, the annular air bladder 146 can expand and exert force on the finger and restrict arterial volume within the finger. In other examples, air bladder 146 can adopt other structures and/or shapes.

Plethysmographic sensor 150 is configured to sense arterial volume of arteries of patient 180 within a sensing region. The sensing region can includes at least a portion of the clamped volume that is clamped by air bladder 146. At least one artery passes through the sensing region. Arteries of patient 180 pulsate during normal blood flow, expanding (with systolic pressure) and relaxing (with diastolic pressure) in volume over the course of each heartbeat cycle. Plethysmographic sensor 150 can sense the expansion and contraction of arteries within the sensing region during arterial pulsation. Plethysmographic sensor 150 is electronically connected to processor 112, such that in operation processor 112 can control the operation of plethysmographic sensor 150 and/or plethysmographic sensor 150 can provide plethysmographic signals to processor 112 representative of arterial volume.

In the depicted example, plethysmographic sensor 150 is a photoplethysmographic sensor including a light emitter and a light sensor. The light emitter is structured and configured to emit light through arteries of patient 180 and the light sensor is configured to receive light emitted by the light emitter after the light has passed through arteries of patient 180. The light emitter can be, for example, an LED. The relative intensity of the received light signal is inversely proportional to arterial volume within the sensing region, such that reduced signal corresponds to increased arterial volume and increased signal corresponds to decreased arterial volume. However, in other examples, plethysmographic sensor 150 can be another suitable sensor for measuring arterial volume.

Air pressure controller 106 is pneumatically connected to air bladder 146 and is configured to adjust the air pressure of the air bladder 146. Air pressure controller 106 can include one or more valves, pumps, pressurized air sources, and/or other pneumatic components capable of selectively causing air to flow to air bladder 146 or out of air bladder 146 to adjust the air pressure inside of air bladder 146. Air pressure controller 106 also includes one or more pressure sensors for sensing the pressure of air within air bladder 146 as well as one or more electronic components for receiving signals from and sending signals to processor 112, such as one or more electronic circuits or controllers. For example, the electronic components of air pressure controller 106 can be configured to receive control signals from processor 112 and/or to send pressure readings from the pressure sensor to processor 112.

Air pressure controller 106 can be operated to cause pressurized air to flow into air bladder 146 in order to increase the air pressure within air bladder 146 and/or to allow air to flow out of air bladder 146 to decrease the air pressure within air bladder 146. For example, air pressure controller 106 can control the position of one or more valves disposed between a source of pressurized air and air bladder 146 to selectively allow air to flow from the pressurized air source to the interior of air bladder 146. Air pressure controller 106 can also be structured and/or configured to control the position of a vent valve to selectively allow air to flow out of air bladder 146. As a further example, air pressure controller 106 can include one or more pumps for compressing air that can be flowed to the interior of air bladder 146. As described previously, the air bladder 146 is deformable such that the air pressure within air bladder 146 can be varied to adjust the force exerted by air bladder 146 on the appendage circumferentially-surrounded by air bladder 146.

Arterial monitor 102 is configured to control operation of air pressure controller 106, such that arterial monitor 102 can cause air pressure controller 106 to adjust the air pressure of air bladder 146. As depicted in FIG. 1, memory 114 includes pressure control module 120. Pressure control module 120 includes one or more executable programs that, when executed, cause processor 112 to cause air pressure controller 106 to adjust the air pressure of air bladder 146. The programs of pressure control module 120 can allow for the air pressure of air bladder 146 to be adjusted to a particular pressure value and/or according to one or more patterns. The patterns can include, for example, a continuous or segmented pressure ramp, as will be discussed in more detail subsequently.

Air pressure controller 106 and plethysmographic sensor 150 are electronically-connected to arterial monitor 102, allowing processor 112 to send signals to and receive signals from both air pressure controller 106 and plethysmographic sensor 150. As will be explained in more detail, air pressure controller 106 can be configured to adjust the pressure of air bladder 146 based on the plethysmographic signals from plethysmographic sensor 150. Processor 112 can receive plethysmographic signals from plethysmographic sensor 150 and cause air pressure controller 106 to adjust the air pressure of air bladder 146 according to the received signals. Processor 112 can also adjust receive pressure signals from air pressure controller 106 describing the air pressure of air bladder 146.

In operation, non-invasive sensor 104 can be used to measure the arterial pressure of patient 180 by combined operation of plethysmographic sensor 150 and air pressure controller 106. Specifically, processor 112 can control operation of air pressure controller 106 according to received arterial volume data from plethysmographic sensor 150 and continuously adjust the air pressure within air bladder 146 to mechanically oppose arterial volume changes within the clamped volume, thereby allowing air pressure controller 106 to cause the arterial volume of arteries within the clamped volume of air bladder 146 to remain constant or substantially constant. For example, air pressure controller 106 can include a proportional-integral-derivative (PID) controller that can be used to control the pressure of air bladder 146 according to the signal from plethysmographic sensor 150. Air pressure controller 106 can receive plethysmographic signals directly from plethysmographic sensor 150 in these examples. Additionally and/or alternatively, pressure control module 120 of memory 114 can include one or more programs that enables processor 112 to function as a PID controller and cause air pressure controller 106 to adjust the air pressure of air bladder 146 to maintain a constant or near constant plethysmographic signal from plethysmographic sensor 150. The pressure required to maintain a constant or near arterial volume of arteries within the volume clamped by air bladder 146 represents the arterial pressure of patient 180, and air pressure controller 106 can provide that pressure signal to arterial monitor 102. The pressure signal can be processed by processor 112 and one or more programs of memory 114, and/or can be presented to a user by user interface 116. An arterial pressure measurement using air pressure controller 106 and plethysmographic sensor 150 by clamping an arterial volume in a patient appendage at a constant or substantially constant value is referred to herein as a “volume clamp” measurement.

The plethysmographic signal intensity or arterial volume selected to be maintained during a volume clamp measurement made using non-invasive sensor 104 can be referred to as the plethysmographic or arterial volume “setpoint.” This setpoint ideally corresponds to a relaxed arterial volume (i.e., undilated by arterial pulsations and unstressed or minimally-stressed by mechanical forces from air bladder 146) within the clamped volume for present conditions of the clamped volume, e.g., including patient appendage position/posture and blood perfusion. In practice, the relaxed arterial volume of the appendage of patient 180 within the clamped volume can be difficult to estimate. As a result, the setpoint used for volume clamp measurements is often offset from the relaxed arterial volume, leading to potentially inaccurate arterial pressure measurements made using hemodynamic sensing system 100.

Non-invasive sensor 104 can also be used to sense arterial volume fluctuations during blood flow. Processor 112 can execute programs of pressure control module 120 to cause air pressure controller 106 to hold the air pressure of air bladder 146 at a constant value. Processor 112 can receive plethysmographic signals representative of arterial volume from plethysmographic sensor 150 while air bladder 146 is held at the constant volume.

The plethysmographic signals measured during constant-volume operation can be used to calibrate the setpoint used for volume clamp measurements. The constant-pressure arterial volume measurements used to calibrate the setpoint are referred to herein as “calibration measurements.” Volume clamp measurements can be interrupted periodically to perform calibration measurements to adjust the setpoint to compensate for small changes patient condition (e.g., patient physiological condition). Calibration measurements can also be performed on a triggered (non-scheduled) basis in response to irregularities indicating that the current setpoint requires recalibration. Each calibration measurement captures plethysmographic data for at least one cardiac cycle. In some examples, multiple plethysmographic waveforms can be collected sequentially for different air pressures of air bladder 146. Collecting multiple plethysmographic waveforms can increase the quantity of plethysmographic data that can be used to adjust the volume clamp setpoint, potentially improving the accuracy of the resultant setpoint following adjustment. In these examples, each air pressure of air bladder 146 is maintained for at least one cardiac cycle, such that the plethysmographic signal collected at each air pressure describes at least one cardiac cycle.

The constant air pressure of air bladder 146 maintained during a calibration measurement can be selected based on the arterial pressures measured during the previous volume clamp measurement. For example, the air pressure can be a ratio of the most recent systolic and diastolic pressures. The ratio can be the midpoint between the systolic and diastolic pressures (e.g., the mean arterial pressure) or any other suitable known ratio. The air pressure selected for a calibration measurement can also be chosen based on another suitable operational need or parameter. In some examples, an air pressure of air bladder 146 maintained during a calibration measurement can be referred to a “calibration air pressure.”

Setpoint adjustment module 130 is stored by memory 114 and includes one or more executable programs that can be executed by processor 112 to analyze plethysmographic signals received from plethysmographic sensor 150 during a calibration measurement and to adjust the setpoint used for volume clamp measurements. Processor 112 can use outputs of the programs of setpoint adjustment module 130 to create calibration factors according to methods disclosed herein, as will be described in more detail subsequently. Processor 112 can store the new setpoint to memory 114 for use with further volume clamp measurements and/or use the updated setpoint to control operation of air pressure controller 106 based on signals from plethysmographic sensor 150.

In some examples, air pressure controller 106 can include one or more logic-capable hardware elements. For example, air pressure controller 106 can include a separate processor, memory, and/or user interface that are substantially similar to processor 112, memory 114, and/or user interface 116, respectively that are able to execute the programs of pressure control module 120 to cause air pressure controller 106 to adjust the air pressure of air bladder 146 to a desired pressure or according to a desired pattern. Further, while arterial monitor 102 and non-invasive sensor 104 are shown as separate elements in FIGS. 1-2, arterial monitor 102 and non-invasive sensor 104 can be formed as separate subcomponents or subsystems of a single device or system capable of performing the methods described herein.

FIG. 2 is a detailed isometric view of non-invasive sensor 104 installed on an appendage of patient 180. FIG. 2 depicts non-invasive sensor 104, including air bladder 146, and patient 180. In FIG. 2, non-invasive sensor 104 also includes cuff housing 220, sensor housing 222, and control line 230. Patient 180 includes finger 240.

Cuff housing 220 is a housing element that extends around air bladder 146, such that air bladder 146 is circumferentially surrounded by cuff housing 220. In the depicted example, cuff housing 220 is annular and air bladder 146 also adopts a generally annular shape. Cuff housing 220 is attached to sensor housing 222. Sensor housing 222 houses sensing components of plethysmographic sensor 150 and, in some examples, sensor housing 222 can also house air pressure controller 106. In some examples, cuff housing 220 also houses components of plethysmographic sensor 150 (e.g., an LED of a photoplethysmographic sensor). Control line 230 includes electronic communication lines for enabling communication between plethysmographic sensor 150 and processor 112 as well as pneumatic channels for channel air to the interior of air bladder 146. In the depicted example, the appendage of patient 180 for which arterial pressure and arterial volume can be measured is finger 240. The clamped volume in the depicted example is the portion of finger 240 that is circumferentially surrounded by air bladder 146. In other examples, cuff housing 220, air bladder 146, and/or other elements of non-invasive sensor 104 can be structured to fit other appendages of a patient that are suitable for sensing hemodynamic data.

The plethysmographic signal intensity (e.g., an amount of light transmitted through the patient appendage) or arterial volume selected to be maintained during a volume clamp measurement made using non-invasive sensor 104 can be referred to as the plethysmographic “setpoint.” This setpoint ideally corresponds to a relaxed arterial volume (i.e., undilated by arterial pulsations and unstressed or minimally-stressed by mechanical forces from air bladder 146) within the clamped volume for present conditions of the clamped volume, e.g., including patient hand position/posture and blood perfusion. In practice, the relaxed arterial volume of the appendage of patient 180 within the clamped volume can be difficult to estimate. As a result, the setpoint used for volume clamp measurements is often offset from the relaxed arterial volume, leading to potentially inaccurate arterial pressure measurements made using hemodynamic sensing system 100.

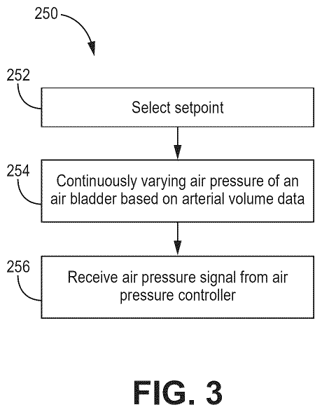

FIG. 3 is a flow diagram of method 250, which is a high-level illustration of a method of performing a volume clamp measurement. Method 250 includes steps 254-256 of continuously varying air pressure of an air bladder based on arterial volume data (step 254), and receiving an air pressure signal from an air pressure controller (step 256).

In step 254, the air pressure of air bladder 146 is varied continuously by air pressure controller 106 based on arterial volume data while air bladder 146 surrounds an appendage of patient 180 (e.g., finger 240). The arterial volume data can be, for example, plethysmographic signals from plethysmographic sensor 150. More specifically, processor 112 can receive plethysmographic signals representative of arterial volume of the clamped volume from plethysmographic sensor 150 and cause air pressure controller 106 to vary the pressure of air bladder 146 to maintain a plethysmographic signal setpoint. The setpoint can be set manually (e.g., by user input at user interface 116) or determined according to another method described herein.

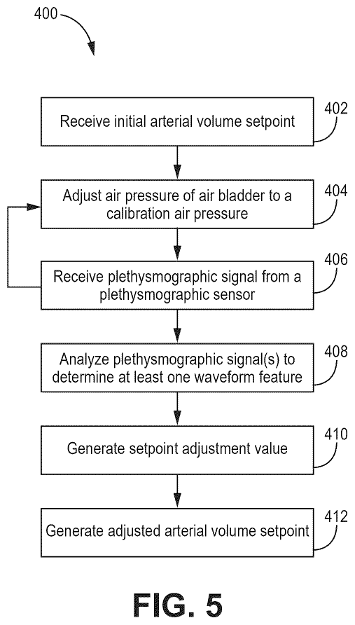

In step 256, an air pressure signal is received from air pressure controller 106. As the pressure of air bladder 146 is continuously varied, air pressure controller 106 can transmit a signal to processor 112 that is representative of the pressure of air bladder 146. As described previously, this signal represents an arterial pressure waveform. As also described previously, a volume clamp measurement taken at a setpoint that does not accurately estimate the unstressed volume of arteries in the sensing region can reduce the accuracy of pressure measurements made using method 350 and further can reduce the amplitude of the measured arterial pressure waveform(s). Method 400, discussed subsequently with respect to FIG. 5, provides more accurate volume clamp setpoints than other, existing methods of setpoint determination.

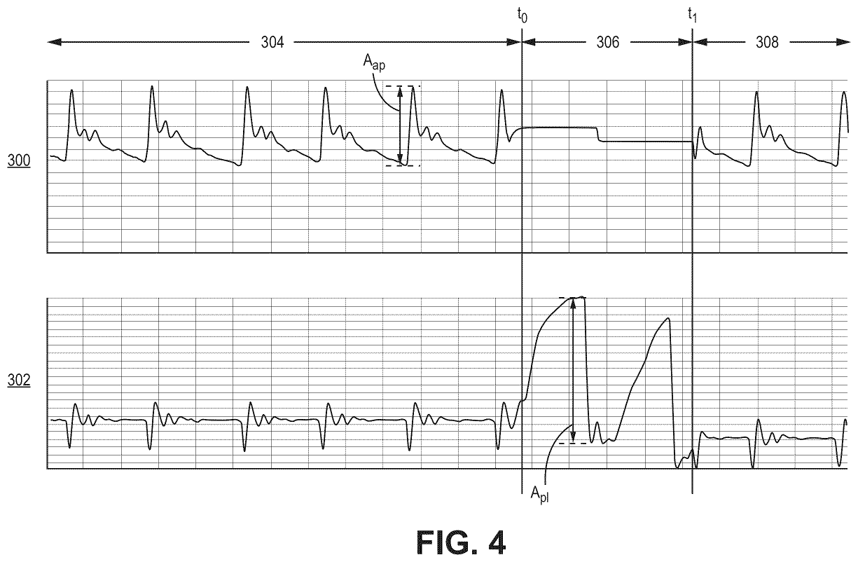

FIG. 4 is a parallel graph of air pressure signals 300 and plethysmographic signals 302 as a function of time in an illustrative example of an operation period of hemodynamic sensing system 100. FIG. 4 illustrates data collected by hemodynamic sensing system 100 through the end of first volume clamp measurement 304, transitioning at time to into calibration measurement 306, then into second volume clamp measurement 308 at time t₁. Second volume clamp measurement 308 is performed at an updated plethysmographic setpoint determined based on the waveforms collected during calibration measurement 306. As described above with reference to FIGS. 1 and 2, hemodynamic sensing system 100 maintains a constant or near constant arterial volume (i.e., a setpoint) during a volume clamp measurement according to plethysmographic signals received from plethysmographic sensor 150. The air pressure of air bladder 146 required to maintain the setpoint represents the arterial pressure of patient 180. Accordingly, during volume clamp measurements 304 and 308, the value of air pressure signals 300 varies and the value of plethysmographic signals 302 is constant or substantially constant. Also as described previously, hemodynamic sensing system 100 maintains one or more constant air pressures of air bladder 146 during a calibration measurement and measures arterial volume during the calibration measurement. Accordingly, during calibration measurement 306, plethysmographic signals 302 vary and air pressure signals 300 remain constant or substantially constant. In the depicted example, two air pressure values are used during calibration measurement 306 and plethysmographic data for a complete cardiac cycle is collected for each air pressure. Air pressure signals 300 taken during volume clamp measurements 304 and 308 are representative of arterial pressure waveforms and plethysmographic signals 302 taken during calibration measurement 306 are representative of arterial volume waveforms.

First volume clamp measurement 304 is performed at a first setpoint value. The setpoint can be preselected and/or can be based on a previous calibration measurement not depicted in FIG. 4. At time to, calibration measurement 306 is performed. The air pressures maintained during calibration measurement 306 are selected based on the air pressure signals 300 collected during first volume clamp measurement 304. Processor 112 can use one or more programs of pressure control module 120 and/or setpoint adjustment module 130 to determine the air pressure(s) used for calibration measurement 306 based on air pressure signals 300 collected during first volume clamp measurement 304. Following calibration measurement 306, second volume clamp measurement 308 is performed at time t₁. Second volume clamp measurement 308 is performed at a second setpoint that is different from the setpoint used during first volume clamp measurement 304. The second setpoint is based on the plethysmographic signals 302 collected during calibration measurement 306. As will be described in more detail subsequently, the setpoint can be based on peak shape, amplitude, or another suitable factor of an arterial pressure waveform, or any combination thereof. In the depicted example, the second setpoint corresponds to a lower plethysmographic signal than the first setpoint used during first volume clamp measurement 304. In the depicted example, plethysmographic signals 302 are collected using a photoplethysmographic sensor (e.g., plethysmographic sensor 150), such that lower values of plethysmographic signals 302 correspond to a greater arterial volume. Accordingly, the second setpoint corresponds to an average clamp pressure applied by air bladder 146 that is lower than the average clamp pressure applied to maintain the first setpoint.

As referred to herein, a waveform “amplitude” refers to a peak-to-trough amplitude of a waveform. FIG. 4 illustrates pressure amplitude Aₐₚ and plethysmographic amplitude Aₚₗ, which are peak to trough amplitudes of arterial pressure and arterial volume waveforms, respectively. Pressure amplitude Aₐₚ represents a peak-to-trough amplitude of a waveform of air pressure signals 300 taken during first volume clamp measurement 304. Plethysmographic amplitude Aₚₗ represents a peak-to-trough amplitude of a waveform represented by plethysmographic signals 302 during calibration measurement 306. Pressure amplitude Aₐₚ is one example of an amplitude of an arterial pressure waveform represented by air pressure signals 300 and, similarly, plethysmographic amplitude Aₚₗ is one example an amplitude of an arterial volume waveform represented by plethysmographic signals 302. In other examples, other amplitude values are possible.

The shape of plethysmographic signals 302 during calibration measurement 306 can be used to determine how close the air pressure(s) of air bladder 146 are to the patient's MAP. Plethysmographic signals measured at air bladder pressures below the MAP of arteries generally have low amplitudes, as the low pressure of air bladder 146 does not exert enough force on the arterial volume to cause the arterial volume to decrease significantly during diastole. Similarly, as the pressure of air bladder 146 is increased above the MAP, the force applied by air bladder 146 reduces the variation in volume during arterial pulsation. More specifically, at pressures above the patient's MAP, the force exerted by air bladder 146 can significantly reduce arterial expansion during systole and create rounded plethysmographic peaks during diastole (i.e., by applying excessive pressure during arterial contraction). Air pressures of air bladder 146 that are similar or equal to the patient's actual MAP result in a plethysmographic waveforms that have high amplitudes and that do not have rounded diastolic peaks. The plethysmographic waveform can be analyzed at multiple pressures and, as will be explained in more detail subsequently (and particularly with respect to method 400; FIG. 5), the setpoint used for volume clamp measurements can be adjusted according to the shape, amplitude, and/or other parameters of the plethysmographic waveform.

Using a setpoint in a volume clamp measurement 304, 308 that accurately approximates the unstressed volume of the clamped arterial volume in turn improves the accuracy with which the pressure signal 300 from a volume clamp measurement 304, 308 approximates the patient's arterial pressure. The unstressed state of an artery represents the state of the artery in which the transmural pressure over the arterial wall is zero, such that the intravascular pressure is equal or substantially equal to the pressure exerted on the outside of the arterial wall. Accordingly, in the unstressed state, the air pressure of air bladder 146 required to maintain the unstressed volume accurately represents the blood pressure within the clamped arterial volume. Notably, when the setpoint underestimates arterial volume (i.e., a setpoint at an improperly high plethysmographic value), such that the mechanical force applied by air bladder 146 stresses the artery during a volume clamp measurement, the force applied by air bladder 146 reduces amplitude of the arterial pressure waveforms represented by air pressure signals 300 and results in arterial pressure measurements that overestimate patient blood pressure. Similarly, a setpoint that overestimates the unstressed arterial volume (i.e., a setpoint at an improperly low plethysmographic value), such that the force applied by the artery or arteries to air bladder 146 is greater than the force applied by air bladder 146 to the artery or arteries, also reduces the amplitude of the arterial pressure waveforms represented by air pressure signals 300. Setpoints that overestimate unstressed arterial volume can also result in arterial pressure measurements that underestimate patient blood pressure.

Existing methods of setpoint determination calculate a new setpoint based only on arterial volume data collected during a calibration measurement. Notably, these existing methods can repeatedly underestimate the plethysmographic signal value that corresponds to the unstressed arterial state (i.e., existing methods can overestimate the arterial volume corresponding to the unstressed state). Specifically, as the air pressure selected for calibration measurements (e.g., calibration measurement 306) is selected based on the arterial pressures measured in the previous volume clamp measurement (e.g., volume clamp measurements 304, 308), a volume clamp measurement made at an improperly low plethysmographic setpoint value can result in subsequent setpoints also being underestimated, as volume clamp measurements made at overly low plethysmographic setpoint values often result in erroneously low arterial pressure measurements.

FIG. 5 is a flow diagram of method 400, which is a method of determining plethysmographic setpoint according to the present disclosure. As will be discussed in more detail subsequently, method 400 determines setpoint based on both the previous setpoint and waveform characteristics of one or more arterial volume waveform collected during a calibration measurement. Method 400 includes steps of receiving an initial arterial volume setpoint (step 402), adjusting the air pressure of an air bladder to a calibration air pressure (step 404), receiving a plethysmographic signal from a plethysmographic sensor (step 406), analyzing the plethysmographic signal to determine at least one waveform feature (step 408), generating a setpoint adjustment value based on the first waveform feature (step 410), and generating an adjusted arterial volume setpoint (step 412).

In step 402, an initial arterial volume setpoint is received. The initial arterial volume setpoint is the setpoint value used in a previous volume clamp measurement. The initial arterial volume setpoint is received by processor 112 and can be, for example, recalled from memory 114, among other options.

Steps 404-406 describe a calibration measurement (e.g., calibration measurement 306; FIG. 4). In step 404, the air pressure of air bladder 146 is adjusted to a calibration air pressure. The calibration air pressure can be selected based on the arterial pressures measured during a prior volume clamp measurement or selected based on user input at user interface 116, among other options. Processor 112 can cause air pressure controller 106 to adjust the air pressure of air bladder 146 to the selected calibration air pressure and to maintain the calibration air pressure during subsequent step 406.

In step 406, a plethysmographic signal is received from plethysmographic sensor 150 while the air pressure of the air bladder is at the calibration air pressure. The plethysmographic signal is representative of an arterial volume waveform and describes the volume of the artery or arteries of the appendage surrounded by air bladder 146 for at least one cardiac cycle. The plethysmographic signal can be received by processor 112 and can be stored to memory 114 for use with subsequent steps of method 400.

As illustrated in FIG. 5, steps 404-406 can be repeated multiple times to acquire plethysmographic data at multiple air pressures. At each air pressure of air bladder 146, plethysmographic signal data representing an entire cardiac cycle is collected by plethysmographic sensor 150 and transmitted to processor 112.

In step 408, the plethysmographic signal received in step 406 is analyzed to determine at least one waveform feature. As referred to herein, a “waveform feature” refers to any classifiable feature of a waveform, such as a waveform amplitude, a peak sharpness, a waveform period, or any other suitable feature of an arterial volume waveform. The waveform feature determined in step 408 can be used in subsequent steps of method 400 to adjust the plethysmographic setpoint used for subsequent volume clamp measurement(s). In examples where steps 404-406 are repeated multiple times, such that multiple plethysmographic signals are received by processor 112, multiple waveform features can be determined in step 408. In some examples, a single waveform feature (e.g., a ratio) can be determined from the multiple plethysmographic signals.

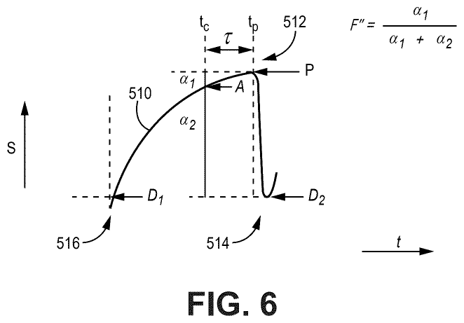

As one specific example, the waveform feature can be a sharpness of the diastolic peak of the waveform represented by the plethysmographic signal. FIG. 6 is a graph illustrating a plethysmographic waveform 510, which is a plethysmographic signal for a single cardiac cycle. FIG. 6 also illustrates values that can be used to create an amplitude ratio for describing diastolic peak sharpness. Axis S represents plethysmographic signal intensity and axis t represents time. Each period of the waveform of plethysmographic waveform 510 represents a single cardiac cycle. Peak 512 is representative of the diastole of the cardiac cycle and trough 514 is representative of the systole of the cardiac cycle. Plethysmographic signal has value P at peak 512 and value D₂ at trough 514. Peak P occurs at time tₚ. Time t_{c} is a time prior to time tₚ and is offset from time t_{c} by peak time offset T. Amplitude A is the peak to trough amplitude of plethysmographic waveform 510 for the represented cardiac cycle. FIG. 6 also depicts previous trough 516, which has signal S value D₁, for clarity and context. However, only one period of the waveform represented by plethysmographic waveform 510 is required to calculate an amplitude ratio.

Value a₁ is the difference of signal value S of plethysmographic waveform 510 at times tₚ and t_{c} and can be obtained by subtracting the signal value S of plethysmographic waveform 510 at time t_{c} from the signal value S of plethysmographic waveform 510 at time tₚ according to the following equation:

$${a}_{1} = P - S ( {t}_{c} ) \qquad [ Equation 1 ]$$

where the function S(t) describes the signal value S of plethysmographic waveform 510 over time t, such S(t_{c}) is the value of signal S at time t_{c}, and where P is the value of signal S at peak 512.

Value a₂ is difference between the value of signal S of plethysmographic waveform 510 at time t_{c} and at trough D₂, and can be obtained by subtracting the value of signal S of waveform 510 at trough D₂ from the value of signal S of at waveform 510 at time t_{c} according to the following equation:

$${a}_{2} = S ( {t}_{c} ) - {D}_{2} \qquad [ Equation 2 ]$$

where S(t_{c}) is the value of signal S of plethysmographic waveform 510 at time t_{c} and D₂ is the value S of plethysmographic waveform 510 at trough 514.

Accordingly, values a₁ and a₂ are related to amplitude A according to the following equation:

$$A = {a}_{1} + {a}_{2} \qquad [ Equation 3 ]$$

which allows calculation of either of a₁ and a₂ from the other of a₁ and a₂ and the peak-to-trough amplitude A of plethysmographic waveform 510 during the cardiac cycle.

Once a₁ and a₂ are known, peak ratio F″ can be calculated according to the following equation:

$${F}^{″} = \frac{{a}_{1}}{{a}_{1} + {a}_{2}} \qquad [ Equation 4 ]$$

For a given waveform, a large value of F″ corresponds to a sharp diastolic peak and a low value of F″ corresponds to a blunt or rounded diastolic peak. Accordingly, the value of F″ can be used to estimate whether the air pressure of air bladder 146 used for the calibration measurement is above the patient's MAP, as rounded diastolic peaks are generally measured at air pressures of air bladder 146 that are above the patient's MAP.

Each waveform collected during a calibration measurement can be analyzed to identify a peak and a trough, corresponding to the diastole and systole of the cardiac cycle, respectively. Time t_{c} can be identified based on the time tₚ at which the peak occurred and based on time differential T (i.e., by subtracting T from time tₚ). Values a₁ and a₂ can be determined for time t_{c} and peak ratio F″ can be calculated therefrom according to equation 4. The peak sharpness of multiple waveforms can be compared by calculating t_{c} for each waveform using the same time differential T. For example, where the calibration measurement collects plethysmographic data for multiple air pressures of air bladder 146, the peak sharpness of the waveform collected at each air pressure can be compared according to the process outlined above by using the same time differential T to calculate t_{c} for each waveform.

Returning to step 408 of method 400 (FIG. 5), the waveform feature can also be an amplitude ratio of two waveforms collected during the calibration measurement. For example, where the calibration measurement collects plethysmographic data for multiple air pressures of air bladder 146, the peak-to-trough amplitudes (e.g., amplitude A; FIG. 6) of each waveform can be compared. As the highest arterial volume waveform amplitudes are generally collected at air pressures of air bladder 146 near or equal to the patient's MAP (as described previously and particularly with reference to the discussion of FIG. 4), comparison of amplitudes can be used to determine which air pressure of air bladder 146 used during the calibration measurement is closer to the patient's MAP.

In step 410, a setpoint adjustment value is generated based on the waveform feature or waveform features determined in step 408. In some examples, only one waveform feature is used to generate the setpoint adjustment value. In further examples, two or more waveform features can be used to generate the setpoint adjustment value. The setpoint adjustment value generated in step 410 can be used to adjust the setpoint used in the previous volume clamp measurement in order to generate a new, adjusted arterial volume setpoint that can be used in a subsequent volume clamp measurement.

In step 412, the previous arterial volume setpoint is adjusted based on the setpoint adjustment value generated in step 410 in order to generate the adjusted arterial volume setpoint. The adjusted arterial volume setpoint corresponds to a plethysmographic signal intensity that can be maintained by operation of air pressure controller 106 during a subsequent volume clamp measurement. In some examples, the setpoint adjustment value is an offset value such that the adjusted setpoint can be generated by adding or subtracting the setpoint adjustment value to or from, respectively, the previous arterial volume setpoint. In further examples, the setpoint adjustment value is a scale factor, such that the adjusted setpoint can be generated by multiplying the previous setpoint adjustment value by the setpoint adjustment value. In yet further examples, the setpoint adjustment value can be any other suitable value for adjusting arterial volume setpoint according to any other suitable function or mathematical operation.

In some examples where plethysmographic signals are collected for multiple air pressures of air bladder 146 (i.e., where steps 404-406 of method 400 are repeated for each air pressure), the setpoint adjustment value can be determined in step 410 according to the following pseudocode:

```
amp_ratio = 100 * amp_max / amp_first
if ((amp_ratio > A) AND (Fₐᵥ > B))
  setpoint_adjust = X * amp_max
else
  setpoint_adjust = Y * amp_max
```

where amp_max is the maximum peak-to-trough amplitude collected during the previous calibration measurement, amp_first is the peak-to-trough amplitude of the plethysmographic signal at the first air pressure used (e.g., an air pressure based on the previous volume clamp measurement), Fₐᵥ is an average of F″ for each waveform (i.e., for the waveform collected for each pressure), and setpoint_adjust is the setpoint adjustment value. Where amp_first is an estimate of patient MAP based on the previous volume clamp (i.e., arterial pressure) measurement, amp_ratio reflects the difference in amplitudes between the air pressure yielding the maximum waveform amplitude and the amplitude of the waveform collected at the MAP estimated based on the volume clamp measurement taken at the prior arterial volume setpoint. Accordingly, where amp_ratio exceeds threshold A (indicating that the maximum waveform amplitude is sufficiently large as compared to the amplitude of the air pressure estimated according to measurements based on the prior setpoint) while Fₐᵥ exceeds threshold B (indicating that the average peak sharpness exceeds a minimum threshold), the above pseudocode determines the setpoint adjustment value by multiplying the value of the maximum peak-to-trough amplitude by constant X. Where either amp_ratio does not exceed threshold A or Fₐᵥ does not exceed threshold B, the setpoint adjustment value is determined by multiplying the value of the maximum-peak to trough amplitude by constant Y. The new setpoint can be calculated by adding the value of setpoint_adjust to the value of the previous plethysmographic setpoint. Constant X is generally a positive value, such that where a new air pressure results in increased peak-to-trough amplitude without impacting peak sharpness (according to threshold B), the plethysmographic signal value maintained as the setpoint is increased. This increased plethysmographic setpoint corresponds to a smaller estimated unstressed arterial volume, as described previously and particularly with reference to the discussion of FIGS. 1-3. In other examples, such as where a new air pressure value corresponds to an increased amplitude but negatively impacts peak sharpness, the plethysmographic signal is maintained (i.e., where Y is equal to 1) or is decreased (i.e., where Y is a negative value).

In other examples, other relationships between waveform characteristics can be determined and used to generate a setpoint adjustment value. For example, reinforcement learning algorithms can be used to determine relationships between plethysmographic waveforms obtained from a calibration measurement and improvements to volume clamp setpoint. As a specific example, a reinforcement learning algorithm can be configured to iteratively change volume clamp setpoint in order to maximize the amplitude of an arterial pressure waveform during a volume clamp measurement. The reinforcement learning algorithm can be trained to create a setpoint adjustment function that relates plethysmographic waveform features of a waveform feature class to setpoint adjustment values over multiple training cycles. As referred to herein, a “waveform feature class” refers to a class that encompasses some or all variations of a waveform feature, where each waveform feature belongs to or is a characteristic of a specific waveform. For example, a waveform feature class can refer to amplitude ratios generally, which a waveform feature (e.g., a feature identified in step 408 of method 400; FIG. 5) can refer to a specific amplitude ratio for a specific waveform. For each training cycle, a volume clamp measurement can be performed according to method 250 and then method 400 can be performed, such that, at least in some examples, steps of method 400 are performed by a reinforcement learning algorithm in order to train the reinforcement learning algorithm. Specifically, the reinforcement learning algorithm can be used to perform steps 408-412 of method 400. The training cycles can be used to train the reinforcement learning algorithm to create setpoint adjustment values based on plethysmographic data generated according to steps 404-406 of method 400. Training the reinforcement learning algorithm creates a setpoint adjustment function that can be used to generate setpoint adjustment values based on plethysmographic data created according to steps 404-406. The reinforcement learning algorithm can evaluate the setpoint adjustment function after each training cycle based on the amplitude of the arterial pressure waveform generated by the next volume clamp measurement. The reinforcement learning algorithm can be configured (e.g., according to a reward function) to create a setpoint adjustment function that maximizes the amplitude of arterial pressure waveforms, which provides the advantages described herein previously. After a sufficient number of training cycles has been completed, the setpoint adjustment function can be extracted from the trained reinforcement learning algorithm and used in subsequent calibration measurements to calibration volume clamp setpoint.

Method 400 provides more accurate setpoints than existing methods of calculating volume clamp setpoint. More specifically, by adjusting the previous setpoint rather than calculating a new setpoint, method 400 has a significantly lower likelihood of persistently underestimating or overestimating the unstressed arterial volume. The use of multiple calibration measurements (i.e., by repeating steps 404-406) and/or the use of multiple waveform features to determine the setpoint adjustment value (i.e., in steps 408-410) can further improve the accuracy of the setpoints produced by method 400, but it is understood that examples using a single calibration measurement and a single waveform feature provide significant improvements to setpoint estimation as compared to existing methods.

By providing volume clamp setpoints that more accurately estimate unstressed arterial volume, the methods and systems described herein enable improvements to volume clamp measurements. Accordingly, the methods and systems described herein also enable improvements to the accuracy with which patient blood pressure can be measured by a volume clamp technique. Notably, methods and systems described herein do not require additional sensors to provide improvements to setpoint calculation and arterial pressure measurements. Rather, the hemodynamic sensor used to make the volume clamp measurement (e.g., non-invasive sensor 104) can also be used to make calibration measurements according to the methods disclosed herein.

The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

### Discussion of Detailed Embodiments

The following are non-exclusive descriptions of possible embodiments of the present invention.

A method of measuring arterial pressure using a non-invasive hemodynamic sensor including receiving a first arterial volume setpoint, adjusting an air pressure of an air bladder to a first air pressure, receiving a first plethysmographic signal from a plethysmographic sensor while the air pressure of the air bladder is at the first air pressure, analyzing the first plethysmographic signal to determine a first waveform feature, generating a setpoint adjustment value based on the first waveform feature, and generating a second arterial volume setpoint by adjusting the first arterial volume setpoint based on the setpoint adjustment value. The first arterial volume setpoint and the first plethysmographic signal are received by a processor and the air pressure of the air bladder is adjusted by a pressure controller operatively connected to the processor. The first plethysmographic signal is representative of a first arterial volume waveform.

The method of the preceding paragraph can optionally include, additionally and/or alternatively, any one or more of the following features, configurations and/or additional components:

A method as set forth above, wherein adjusting the first arterial volume setpoint by the setpoint adjustment value comprises adding the first arterial volume setpoint and the setpoint adjustment value such that the second arterial volume setpoint is a sum of the first arterial volume setpoint and the setpoint adjustment value.

A method as set forth above, and further comprising adjusting the air pressure of the air bladder to a second air pressure after receiving the first signal, receiving a second plethysmographic signal while the air pressure of the air bladder is at the second air pressure, and analyzing the second plethysmographic signal to determine a second waveform feature, wherein generating the setpoint adjustment value comprises generating the setpoint adjustment value based on the first waveform feature and the second waveform feature. The air pressure of the air bladder is adjusted by the pressure controller and the second plethysmographic signal representative of a second arterial volume waveform.

A method as set forth above, wherein the first waveform feature is a first amplitude of the first plethysmographic signal and the second waveform feature is a second amplitude of the second plethysmographic signal.

A method as set forth above, wherein generating the setpoint adjustment value comprises comparing the ratio to an amplitude ratio threshold, selecting a modifier value based on the comparison, determining which of the first amplitude and the second amplitude is greater in value, and generating the setpoint adjustment value based on the modifier value and the amplitude of the first and second amplitudes that is greater in value.

A method as set forth above, wherein the setpoint adjustment value is a multiplication product of the modifier value and the amplitude of the first and second amplitudes that is greater in value.

A method as set forth above, wherein analyzing the first arterial volume waveform to determine the first waveform feature comprises analyzing the first plethysmographic signal to identify a peak, the first plethysmographic signal having a first intensity at the peak, analyzing the first plethysmographic signal to identify a trough, the first plethysmographic signal having a second intensity at the trough, selecting a point along the first plethysmographic signal, the point occurring in the first plethysmographic signal prior to the peak and the first plethysmographic signal having a third intensity at the point, determining a first amplitude difference between first intensity and the third intensity, determining a second amplitude difference between third intensity and the second intensity, and generating a shape parameter according to the following formula:

$$S = \frac{{a}_{1}}{{a}_{1} + {a}_{2}}$$

wherein a₁ is the first amplitude difference, a₂ is the second amplitude difference, and S is the shape parameter.

A method as set forth above, wherein generating the setpoint adjustment value comprises generating the setpoint adjustment value based on the shape parameter.

A method as set forth above, and further comprising continuously varying the air pressure of the air bladder to maintain the first arterial volume setpoint based on arterial volume data from the plethysmographic sensor before adjusting the air pressure of the air bladder to the first air pressure and receiving a signal representative of the air pressure of the air bladder while the air pressure is continuously varied to maintain the first arterial volume setpoint. The air pressure is continuously varied by the pressure controller and the signal representative of the air pressure of the air bladder is received from the pressure controller.

A method as set forth above, and further comprising continuously varying the air pressure of the air bladder to maintain the second arterial volume setpoint based on arterial volume data from the plethysmographic sensor before adjusting the air pressure of the air bladder to the first air pressure and receiving a signal representative of the air pressure of the air bladder while the air pressure is continuously varied to maintain the second arterial volume setpoint. The air pressure is continuously varied by the pressure controller and the signal representative of the air pressure of the air bladder is received from the pressure controller.

A method as set forth above, wherein the plethysmographic sensor is a photoplethysmographic sensor.

A method as set forth above, and further comprising training a reinforcement learning algorithm to adjust a training arterial volume setpoint over a plurality of training cycles and extracting a setpoint adjustment function from the trained reinforcement learning algorithm, wherein the setpoint adjustment function relates waveform features of a waveform feature class to setpoint adjustment values, and wherein the reinforcement learning algorithm is configured to adjust the training arterial volume setpoint to maximize an amplitude of a training arterial pressure waveform.

A method as set forth above, wherein each training cycle of the plurality of training cycles comprises continuously varying, by the pressure controller, the air pressure of the air bladder to maintain the training arterial volume setpoint based on arterial volume data from the plethysmographic sensor, receiving, from the pressure controller, a training signal representative of the air pressure of the air bladder while the air pressure is continuously varied to maintain the training arterial volume setpoint, the training signal representative of the training arterial pressure waveform, selecting an air pressure based on the training arterial pressure waveform, adjusting, by the pressure controller, the air pressure of the air bladder to the selected air pressure, receiving, by the processor, a training plethysmographic signal from the plethysmographic sensor while the air pressure of the air bladder is at the selected air pressure, the training plethysmographic signal representative of a training arterial volume waveform, and adjusting the training arterial volume setpoint based on the training arterial volume waveform.

A system for measuring arterial pressure includes a plethysmographic sensor configured to sense arterial volume, a pressure controller pneumatically connected to an air bladder and configured to adjust an air pressure of the airbladder, a processor in operable communication with the pressure controller and the plethysmographic sensor, and a memory encoding instructions. The instructions, when executed, cause the processor to receive a first arterial volume setpoint, cause the pressure controller to adjust an air pressure of the air bladder to a first air pressure, receive a first plethysmographic signal from the plethysmographic sensor while the air bladder is at the first air pressure, analyze the first plethysmographic signal to determine a first waveform feature, generate a setpoint adjustment value based on the first waveform feature, and generate a second arterial volume setpoint by adjusting the first arterial volume setpoint by the setpoint adjustment value. The first plethysmographic signal is representative of a first arterial volume waveform while the air pressure of the air bladder is at the first air pressure.

The system of the preceding paragraph can optionally include, additionally and/or alternatively, any one or more of the following features, configurations and/or additional components:

A system as set forth above, wherein the instructions, when executed, cause the processor to generate the second arterial volume setpoint by adding the first arterial volume setpoint and the setpoint adjustment value, such that the second arterial volume setpoint is a sum of the first arterial volume setpoint and the setpoint adjustment value.

A system as set forth above, wherein the instructions, when executed, further cause the processor to cause the pressure controller to adjust the air pressure of the air bladder to a second air pressure after receiving the first signal, receive a second plethysmographic signal representative of a second arterial volume waveform while the air pressure of the air bladder is at the second air pressure, and analyze the second plethysmographic signal to determine a second waveform feature; and generate the setpoint adjustment value based on the first waveform feature and the second waveform feature.

A system as set forth above, wherein the first waveform feature is a first amplitude of the first plethysmographic signal and the second waveform feature is a second amplitude of the second plethysmographic signal.

A system as set forth above, wherein the instructions, when executed, cause the processor to generate a ratio of the first amplitude and the second amplitude, compare the ratio to an amplitude ratio threshold, select a modifier value based on the comparison, determine which of the first amplitude and the second amplitude is greater in value, and generate the setpoint adjustment value based on the modifier value and the amplitude of the first and second amplitudes that is greater in value.

A system as set forth above, wherein the setpoint adjustment value is a product of the modifier value and the amplitude of the first and second amplitudes that is greater in value.

A system as set forth above, wherein the instructions, when executed, further cause the processor to analyze the first plethysmographic signal to identify a peak, the first plethysmographic signal having a first intensity at the peak, analyze the first plethysmographic signal to identify a trough, the first plethysmographic signal having a second intensity at the trough, select a point along the first plethysmographic signal, the point occurring in the plethysmographic signal prior to the peak and the first plethysmographic signal having a third intensity at the point, determine a first amplitude difference between first intensity and the third intensity, determine a second amplitude difference between third intensity and the second intensity, and generate the setpoint adjustment value based on the shape parameter.

A system as set forth above, wherein the shape parameter is generated according to the following formula:

$$S = \frac{{a}_{1}}{{a}_{1} + {a}_{2}}$$

wherein a₁ is the first amplitude difference, a₂ is the second amplitude difference; and S is the shape parameter.

A system as set forth above, wherein the instructions, when executed, further cause the processor to cause the pressure controller to continuously vary the air pressure of the air bladder to maintain the second arterial volume setpoint based on arterial volume data from the plethysmographic sensor and receive, from the pressure controller, a signal representative of the air pressure of the air bladder while the air pressure is continuously varied to maintain the second arterial volume setpoint.

A system as set forth above, wherein the instructions, when executed, further cause the processor to cause the pressure controller to continuously vary the air pressure of the air bladder to maintain the first arterial volume setpoint based on arterial volume data from the plethysmographic sensor and receive, from the pressure controller, a signal representative of the air pressure of the air bladder while the air pressure is continuously varied to maintain the first arterial volume setpoint.

A system as set forth above, wherein the plethysmographic sensor is a photoplethysmographic sensor.

The above method(s) can be performed on a living animal or on a simulation, such as on a cadaver, cadaver heart, anthropomorphic ghost, simulator (e.g., with body parts, heart, tissue, etc. being simulated), etc.

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

1. A method of measuring arterial pressure using a non-invasive hemodynamic sensor, the method comprising:
receiving, by a processor, a first arterial volume setpoint;
adjusting, by a pressure controller operatively connected to the processor, an air pressure of an air bladder to a first air pressure;
receiving, by the processor, a first plethysmographic signal from a plethysmographic sensor while the air pressure of the air bladder is at the first air pressure, the first plethysmographic signal representative of a first arterial volume waveform;
analyzing the first plethysmographic signal to determine a first waveform feature;
generating a setpoint adjustment value based on the first waveform feature; and
generating a second arterial volume setpoint by adjusting the first arterial volume setpoint based on the setpoint adjustment value.

2. The method of claim 1, wherein adjusting the first arterial volume setpoint by the setpoint adjustment value comprises adding the first arterial volume setpoint and the setpoint adjustment value such that the second arterial volume setpoint is a sum of the first arterial volume setpoint and the setpoint adjustment value.

3. The method of claim 1, and further comprising:
adjusting, by the pressure controller, the air pressure of the air bladder to a second air pressure after receiving the first signal;
receiving a second plethysmographic signal while the air pressure of the air bladder is at the second air pressure, the second plethysmographic signal representative of a second arterial volume waveform; and
analyzing the second plethysmographic signal to determine a second waveform feature;
wherein generating the setpoint adjustment value comprises generating the setpoint adjustment value based on the first waveform feature and the second waveform feature.

4. The method of claim 3, wherein the first waveform feature is a first amplitude of the first plethysmographic signal and the second waveform feature is a second amplitude of the second plethysmographic signal.

5. The method of claim 4, wherein generating the setpoint adjustment value comprises:
generating a ratio of the first amplitude and the second amplitude;
comparing the ratio to an amplitude ratio threshold;
selecting a modifier value based on the comparison;
determining which of the first amplitude and the second amplitude is greater in value; and
generating the setpoint adjustment value based on the modifier value and the amplitude of the first and second amplitudes that is greater in value.

6. The method of claim 5, wherein the setpoint adjustment value is a multiplication product of the modifier value and the amplitude of the first and second amplitudes that is greater in value.

7. The method of claim 1, wherein:
analyzing the first arterial volume waveform to determine the first waveform feature comprises:
analyzing the first plethysmographic signal to identify a peak, the first plethysmographic signal having a first intensity at the peak;
analyzing the first plethysmographic signal to identify a trough, the first plethysmographic signal having a second intensity at the trough;
selecting a point along the first plethysmographic signal, the point occurring in the first plethysmographic signal prior to the peak and the first plethysmographic signal having a third intensity at the point;
determining a first amplitude difference between first intensity and the third intensity;
determining a second amplitude difference between third intensity and the second intensity;
generating a shape parameter according to the following formula:
$$S = \frac{{a}_{1}}{{a}_{1} + {a}_{2}}$$
wherein:
a₁ is the first amplitude difference;
a₂ is the second amplitude difference; and
S is the shape parameter; and
generating the setpoint adjustment value comprises generating the setpoint adjustment value based on the shape parameter.

8. The method of claim 1, and further comprising:
continuously varying, by the pressure controller, the air pressure of the air bladder to maintain the second arterial volume setpoint based on arterial volume data from the plethysmographic sensor; and
receiving, from the pressure controller, a signal representative of the air pressure of the air bladder while the air pressure is continuously varied to maintain the second arterial volume setpoint.

9. The method of claim 1, and further comprising:
continuously varying, by the pressure controller, the air pressure of the air bladder to maintain the first arterial volume setpoint based on arterial volume data from the plethysmographic sensor before adjusting the air pressure of the air bladder to the first air pressure; and
receiving, from the pressure controller, a signal representative of the air pressure of the air bladder while the air pressure is continuously varied to maintain the first arterial volume setpoint.

10. The method of claim 1, and further comprising:
training a reinforcement learning algorithm to adjust a training arterial volume setpoint over a plurality of training cycles, wherein:
the reinforcement learning algorithm is configured to adjust the training arterial volume setpoint to maximize an amplitude of a training arterial pressure waveform; and
each training cycle of the plurality of training cycles comprises:
continuously varying, by the pressure controller, the air pressure of the air bladder to maintain the training arterial volume setpoint based on arterial volume data from a plethysmographic sensor;
receiving, from the pressure controller, a training signal representative of the air pressure of the air bladder while the air pressure is continuously varied to maintain the training arterial volume setpoint, the training signal representative of the training arterial pressure waveform;
selecting an air pressure based on the training arterial pressure waveform;
adjusting, by the pressure controller, the air pressure of the air bladder to the selected air pressure;
receiving, by the processor, a training plethysmographic signal from the plethysmographic sensor while the air pressure of the air bladder is at the selected air pressure, the training plethysmographic signal representative of a training arterial volume waveform; and
adjusting the training arterial volume setpoint based on the training arterial volume waveform; and
extracting a setpoint adjustment function from the trained reinforcement learning algorithm, wherein the setpoint adjustment function relates waveform features of a waveform feature class to setpoint adjustment values;
wherein:
the first waveform feature belongs to the waveform feature class; and
generating the setpoint adjustment value comprises calculating the setpoint adjustment value using the setpoint adjustment function and the first waveform feature.

11. A system for measuring arterial pressure, the system comprising:
a plethysmographic sensor configured to sense arterial volume;
a pressure controller pneumatically connected to an air bladder and configured to adjust an air pressure of the airbladder;
a processor in operable communication with the pressure controller and the plethysmographic sensor; and
a memory encoding instructions that, when executed, cause the processor to:
receive a first arterial volume setpoint;
cause the pressure controller to adjust an air pressure of the air bladder to a first air pressure;
receive a first plethysmographic signal from the plethysmographic sensor while the air pressure of the air bladder is at the first air pressure, the first plethysmographic signal representative of a first arterial volume waveform;
analyze the first plethysmographic signal to determine a first waveform feature;
generate a setpoint adjustment value based on the first waveform feature; and
generate a second arterial volume setpoint by adjusting the first arterial volume setpoint by the setpoint adjustment value.

12. The system of claim 11, wherein the instructions, when executed, cause the processor to generate the second arterial volume setpoint by adding the first arterial volume setpoint and the setpoint adjustment value, such that the second arterial volume setpoint is a sum of the first arterial volume setpoint and the setpoint adjustment value.

13. The system of claim 11, wherein the instructions, when executed, further cause the processor to:
cause the pressure controller to adjust the air pressure of the air bladder to a second air pressure after receiving the first signal;
receive a second plethysmographic signal representative of a second arterial volume waveform while the air pressure of the air bladder is at the second air pressure;
analyze the second plethysmographic signal to determine a second waveform feature; and
generate the setpoint adjustment value based on the first waveform feature and the second waveform feature.

14. The system of claim 13, wherein the first waveform feature is a first amplitude of the first plethysmographic signal and the second waveform feature is a second amplitude of the second plethysmographic signal.

15. The system of claim 14, wherein the instructions, when executed, cause the processor to:
generate a ratio of the first amplitude and the second amplitude;
compare the ratio to an amplitude ratio threshold;
select a modifier value based on the comparison;
determine which of the first amplitude and the second amplitude is greater in value; and
generate the setpoint adjustment value based on the modifier value and the amplitude of the first and second amplitudes that is greater in value.

16. The system of claim 15, wherein the setpoint adjustment value is a product of the modifier value and the amplitude of the first and second amplitudes that is greater in value.

17. The system of claim 11, wherein the instructions, when executed, further cause the processor to:
analyze the first plethysmographic signal to identify a peak, the first plethysmographic signal having a first intensity at the peak;
analyze the first plethysmographic signal to identify a trough, the first plethysmographic signal having a second intensity at the trough;
select a point along the first plethysmographic signal, the point occurring in the plethysmographic signal prior to the peak and the first plethysmographic signal having a third intensity at the point;
determine a first amplitude difference between first intensity and the third intensity;
determine a second amplitude difference between third intensity and the second intensity;
generate a shape parameter according to the following formula:
$$S = \frac{{a}_{1}}{{a}_{1} + {a}_{2}}$$
wherein:
a₁ is the first amplitude difference
a₂ is the second amplitude difference; and
S is the shape parameter; and
generate the setpoint adjustment value based on the shape parameter.

18. The system of claim 11, wherein the instructions, when executed, further cause the processor to:
cause the pressure controller to continuously vary the air pressure of the air bladder to maintain the second arterial volume setpoint based on arterial volume data from the plethysmographic sensor; and
receive, from the pressure controller, a signal representative of the air pressure of the air bladder while the air pressure is continuously varied to maintain the second arterial volume setpoint.

19. The system of claim 11, wherein the instructions, when executed, further cause the processor to:
cause the pressure controller to continuously vary the air pressure of the air bladder to maintain the first arterial volume setpoint based on arterial volume data from the plethysmographic sensor; and
receive, from the pressure controller, a signal representative of the air pressure of the air bladder while the air pressure is continuously varied to maintain the first arterial volume setpoint.

20. The system of claim 11, wherein the plethysmographic sensor is a photoplethysmographic sensor.
